(19) United States
(12) Patent Application Publication
(10) Pub. No.: US 2020/0390505 A1
(43) Pub. Date: Dec. 17, 2020

(54) INTERVENTIONAL MEDICAL DEVICE TRACKING

(71) Applicant: KONINKLIJKE PHILIPS N.V., EINDHOVEN (NL)

(72) Inventors: SHYAM BHARAT, ARLINGTON, MA (US); ALVIN CHEN, CAMBRIDGE, MA (US); KUNAL VAIDYA, BOSTON, MA (US); FRANCOIS GUY GERARD MARIE VIGNON, ANDOVER, MA (US); RAMON QUIDO ERKAMP, SWAMPSCOTT, MA (US); AMEET KUMAR JAIN, BOSTON, MA (US)

(21) Appl. No.: 16/971,030

(22) PCT Filed: Feb. 22, 2019
(86) PCT No.: PCT/EP2019/054399

§ 371 Date: Aug. 19, 2020

Related U.S. Application Data
(60) Provisional application No. 62/633,788, filed on Feb. 22, 2018.

(51) Int. Cl. A61B 34/20; A61B 8/12; A61B 8/00; A61B 8/08; G01S 15/89; G01S 7/52

(52) U.S. Cl. CPC A61B 34/20; A61B 8/12; A61B 8/463; A61B 8/54; A61B 8/0883; A61B 2090/3788; G01S 15/899; G01S 7/5206; A61B 2034/2065; A61B 2034/2063; A61B 8/0841

(57) ABSTRACT

A controller includes a memory that stores instructions, and a processor that executes the instructions. When executed by the processor, the instructions cause the controller to execute a process that includes controlling an imaging probe. The imaging probe is controlled to activate imaging elements to emit imaging signals to generate three or more imaging planes, to simultaneously capture an interventional device and anatomy targeted by the interventional device. The imaging probe is also controlled to simultaneously capture both the interventional device and the anatomy targeted by the interventional device. The imaging probe is controlled to capture at least one of the interventional device and the anatomy targeted by the interventional device in at least two of the three or more imaging planes, and to capture the other of the interventional device and the anatomy targeted by the interventional device in at least one of the three or more imaging planes.

### BACKGROUND

A transesophageal echocardiography (TEE) ultrasound probe is commonly used in cardiac monitoring and navigation. Currently available multi-plane imaging modes for a TEE ultrasound probe include X-plane and full three-dimensional (3D) volume.

Ultrasound tracking technology estimates the position of a passive ultrasound sensor (e.g., PZT, PVDF, copolymer or other piezoelectric material) in the field of view (FOV) of a diagnostic ultrasound B-mode image by analyzing the signal received by the passive ultrasound sensor as the imaging beams of the ultrasound probe sweep the field of view. Time-of-flight measurements provide the axial/radial distance of the passive ultrasound sensor from the imaging array, while amplitude measurements and knowledge of the beam firing sequence provide the lateral/angular position of the passive ultrasound sensor.

FIG. 1 illustrates a known system for tracking an interventional medical device using a passive ultrasound sensor. In FIG. 1, an ultrasound probe 102 emits an imaging beam 103 that sweeps across a passive ultrasound sensor 104 on a tool tip of an interventional medical device 105. An image of tissue 107 is fed back by the ultrasound probe 102. A location of the passive ultrasound sensor 104 on the tool tip of the interventional medical device 105 is provided as a tip location 108 upon determination by a signal processing algorithm. The tip location 108 is overlaid on the image of tissue 107 as an overlay image 109. The image of tissue 107, the tip location 108, and the overlay image 109 are all displayed on a display 100.

### SUMMARY

According to an aspect of the present disclosure, a controller for controlling tracking of an interventional medical device in a patient includes a memory that stores instructions, and a processor that executes the instructions. When executed by the processor, the instructions cause the controller to execute a process that includes controlling an imaging probe. The imaging probe is controlled to activate imaging elements to emit imaging signals to generate three or more imaging planes including a first imaging plane, a second imaging plane, and a third imaging plane perpendicular to the second imaging plane, to simultaneously capture an interventional device and anatomy targeted by the interventional device. The imaging probe is also controlled to simultaneously capture both the interventional device and the anatomy targeted by the interventional device. The imaging probe is controlled to capture at least one of the interventional device and the anatomy targeted by the interventional device in at least two of the three or more imaging planes, and to capture the other of the interventional device and the anatomy targeted by the interventional device in at least one of the three or more imaging planes.

According to another aspect of the present disclosure, a method for tracking an interventional medical device in a patient includes emitting, by activated imaging elements controlled by an imaging probe, imaging signals to generate three or more imaging planes including a first imaging plane, a second imaging plane, and a third imaging plane perpendicular to the second imaging plane, to simultaneously capture an interventional device and anatomy targeted by the interventional device. The method also includes simultaneously capturing the interventional device and the anatomy targeted by the interventional device. The imaging probe is controlled to capture at least one of the interventional device and the anatomy targeted by the interventional device in at least two of the three or more imaging planes, and to capture the other of the interventional device and the anatomy targeted by the interventional device in at least one of the three or more imaging planes.

According to yet another aspect of the present disclosure, a system for tracking an interventional medical device in a patient includes an imaging probe and a controller. The imaging probe is configured to activate imaging elements to emit imaging signals to generate three or more imaging planes including a first imaging plane, a second imaging plane, and a third imaging plane perpendicular to the second imaging plane, to simultaneously capture an interventional device and anatomy targeted by the interventional device. The controller controls the imaging probe to simultaneously capture both the interventional device and the anatomy targeted by the interventional device. The imaging probe is controlled to capture at least one of the interventional device and the anatomy targeted by the interventional device in at least two of the three or more imaging planes, and to capture the other of the interventional device and the anatomy targeted by the interventional device in at least one of the three or more imaging planes. The controller includes a signal processor that processes image signals that simultaneously capture at least one of the interventional device and the anatomy targeted by the interventional device in at least two of the three or more imaging planes and the other of the interventional device and the anatomy targeted by the interventional device in at least one of the three or more imaging planes.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a known system for interventional medical device tracking using a passive ultrasound sensor, in accordance with a representative embodiment.

FIG. 2 is an illustrative embodiment of a general computer system, on which a method of interventional medical device tracking can be implemented, in accordance with a representative embodiment.

FIG. 3 illustrates a method for interventional medical device tracking, in accordance with a representative embodiment.

FIG. 4A illustrates a relationship between a probe and a controller for interventional medical device tracking, in accordance with a representative embodiment.

FIG. 4B illustrates another relationship between a probe and a controller for interventional medical device tracking, in accordance with a representative embodiment.

FIG. 5A illustrates a cross section of a probe for interventional medical device tracking, in accordance with a representative embodiment.

FIG. 5B illustrates a simplified view of imaging planes in the embodiment of FIG. 5A.

FIG. 6A illustrates another cross section of a probe for interventional medical device tracking, in accordance with a representative embodiment.

FIG. 6B illustrates a simplified view of imaging planes in the embodiment of FIG. 6A.

FIG. 7A illustrates another cross section of a probe for interventional medical device tracking, in accordance with a representative embodiment.

FIG. 7B illustrates a simplified view of imaging planes in the embodiment of FIG. 7A.

FIG. 8A illustrates another cross section of a probe for interventional medical device tracking, in accordance with a representative embodiment.

FIG. 8B illustrates a simplified view of imaging planes in the embodiment of FIG. 8A.

FIG. 9 illustrates views presented on a user interface for interventional medical device tracking, in accordance with a representative embodiment.

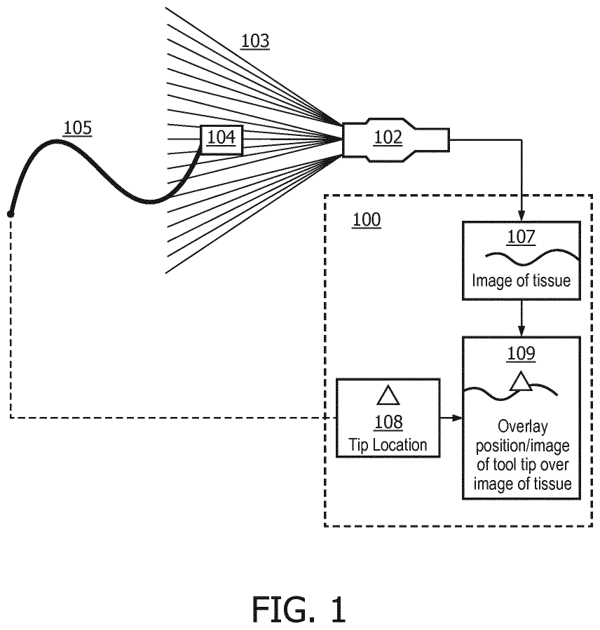

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only, and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As introduced above, use of an X-plane can provide a high frame rate, but only 2 adjustable imaging planes. On the other hand, use of a full three-dimensional (3D) volume can provide control over slicing, but a low frame rate. The present disclosure provides an ability to simultaneously visualize both an interventional medical device and anatomy targeted by the interventional medical device using, for example, the same ultrasound imaging probe by emitting imaging signals in three or more imaging planes. To be clear from the start, the simultaneous emission and capture by the ultrasound imaging probe may involve emitting and capturing the interventional medical device and targeted anatomy when the interventional medical device and targeted anatomy are physically separated in a three-dimensional space.

As described for embodiments below, tissue around a device can be visualized with other quantitative navigation metrics, without losing sight of desired anatomy. Device tracking output can be bootstrapped to an imaging plane selection algorithm, via an automatic feedback/control loop that links device location to control of imaging plane selection. An example of an automatic feedback/control loop is a remote control link (RCL), which tracks an identified device through imaging planes as the device is moved. By linking the interventional device tracking output and the imaging plane selection, multiple different embodiments described herein provide varying capabilities. In other words, the interventional device tracking can be used as part of a feedback loop to ensure that the ability to track the interventional device continues, so that one or more imaging planes can be tied or dedicated to the interventional device. Thus, device tracking can be used to automatically visually follow a device with the imaging planes, in order to continue tracking the interventional device.

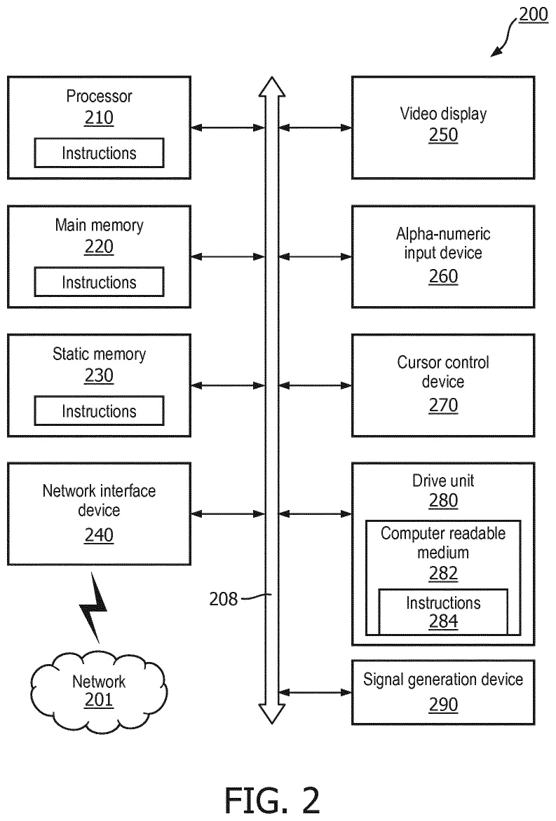

FIG. 2 is an illustrative embodiment of a general computer system, on which a method of interventional medical device tracking can be implemented, in accordance with a representative embodiment. The computer system 200 can include a set of instructions that can be executed to cause the computer system 200 to perform any one or more of the methods or computer based functions disclosed herein. The computer system 200 may operate as a standalone device or may be connected, for example, using a network 201, to other computer systems or peripheral devices.

The computer system 200 can be implemented as or incorporated into various devices, such as a stationary computer, a mobile computer, a personal computer (PC), a laptop computer, a tablet computer, an ultrasound system, an ultrasound probe, or any other machine capable of executing a set of instructions (sequential or otherwise) that specify actions to be taken by that machine. The computer system 200 can be incorporated as or in a device that in turn is in an integrated system that includes additional devices. In an embodiment, the computer system 200 can be implemented using electronic devices that provide voice, video or data communication. Further, while the computer system 200 is illustrated as a single system, the term “system” shall also be taken to include any collection of systems or sub-systems that individually or jointly execute a set, or multiple sets, of instructions to perform one or more computer functions.

As illustrated in FIG. 2, the computer system 200 includes a processor 210. A processor for a computer system 200 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A processor is an article of manufacture and/or a machine component. A processor for a computer system 200 is configured to execute software instructions to perform functions as described in the various embodiments herein. A processor for a computer system 200 may be a general-purpose processor or may be part of an application specific integrated circuit (ASIC). A processor for a computer system 200 may also be a microprocessor, a microcomputer, a processor chip, a controller, a microcontroller, a digital signal processor (DSP), a state machine, or a programmable logic device. A processor for a computer system 200 may also be a logical circuit, including a programmable gate array (PGA) such as a field programmable gate array (FPGA), or another type of circuit that includes discrete gate and/or transistor logic. A processor for a computer system 200 may be a central processing unit (CPU), a graphics processing unit (GPU), or both. Additionally, any processor described herein may include multiple processors, parallel processors, or both. Multiple processors may be included in, or coupled to, a single device or multiple devices.

Moreover, the computer system 200 includes a main memory 220 and a static memory 230 that can communicate with each other via a bus 208. Memories described herein are tangible storage mediums that can store data and executable instructions, and are non-transitory during the time instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A memory described herein is an article of manufacture and/or machine component. Memories described herein are computer-readable mediums from which data and executable instructions can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. Memories may be volatile or non-volatile, secure and/or encrypted, unsecure and/or unencrypted.

As shown, the computer system 200 may further include a video display unit 250, such as a liquid crystal display (LCD), an organic light emitting diode (OLED), a flat panel display, a solid-state display, or a cathode ray tube (CRT). Additionally, the computer system 200 may include an input device 260, such as a keyboard/virtual keyboard or touch-sensitive input screen or speech input with speech recognition, and a cursor control device 270, such as a mouse or touch-sensitive input screen or pad. The computer system 200 can also include a disk drive unit 280, a signal generation device 290, such as a speaker or remote control, and a network interface device 240.

In an embodiment, as depicted in FIG. 2, the disk drive unit 280 may include a computer-readable medium 282 in which one or more sets of instructions 284, e.g. software, can be embedded. Sets of instructions 284 can be read from the computer-readable medium 282. Further, the instructions 284, when executed by a processor, can be used to perform one or more of the methods and processes as described herein. In an embodiment, the instructions 284 may reside completely, or at least partially, within the main memory 220, the static memory 230, and/or within the processor 210 during execution by the computer system 200.

In an alternative embodiment, dedicated hardware implementations, such as application-specific integrated circuits (ASICs), programmable logic arrays and other hardware components, can be constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing can be constructed to implement one or more of the methods or functionality as described herein, and a processor described herein may be used to support a virtual processing environment.

The present disclosure contemplates a computer-readable medium 282 that includes instructions 284 or receives and executes instructions 184 responsive to a propagated signal; so that a device connected to a network 101 can communicate voice, video or data over the network 201. Further, the instructions 284 may be transmitted or received over the network 201 via the network interface device 240.

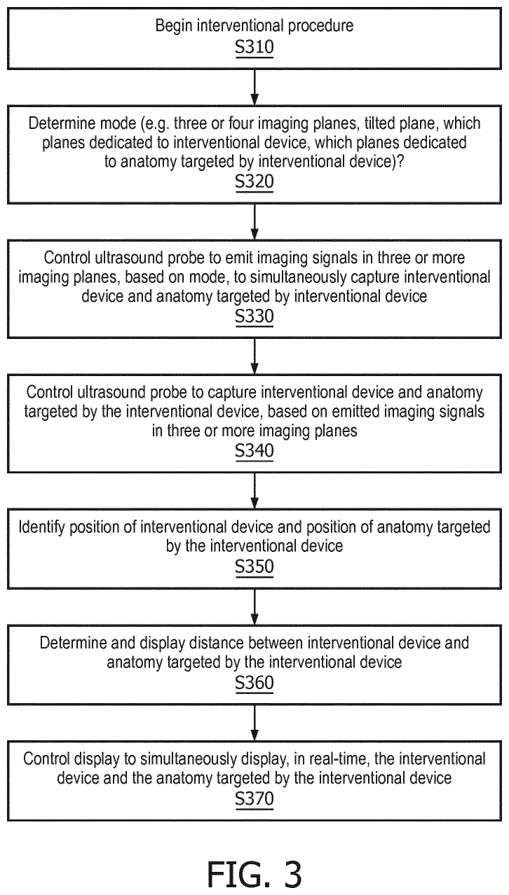

FIG. 3 illustrates a method for interventional medical device tracking, in accordance with a representative embodiment.

In FIG. 3, an interventional procedure begins at S310. An interventional procedure is a procedure in which an interventional medical device is partially or fully placed in the body of a patient, such as for exploratory diagnosis or treatment. An interventional medical device may be or may include a wire, an implant, a sensor including a passive ultrasound sensor, or other forms of tangible devices placed into bodies of patients.

At S320, a mode is determined. A mode may consist of a set of one or more selecting settings such as three or four imaging planes, rotations of planes about an axis, which planes are dedicated to an interventional device, and which planes are dedicated to anatomy targeted by an interventional device. The term “dedicated” as used herein may refer to an assignment of planes to a specific target, which for the purposes of the present disclosure is either an interventional device, or anatomy targeted by the interventional device. The interventional device may be targeted by dedicated planes that track the interventional device in two dimensions or three dimensions as the interventional device moves in the body of the patient.

The anatomy targeted by the interventional device may be designated by a user instruction, such as by using a mouse and cursor or a touch screen. The anatomy may be a specific position on the surface of an organ such as a heart or lung, and may be targeted by the interventional device in the sense that the interventional device is moved towards the anatomy targeted by the interventional device. The interventional device may also be designated by a user, but may alternatively be automatically identified and tracked, such as with the use of a sensor made of a specific material that is readily identified in ultrasound.

At S330, an ultrasound probe is controlled to emit imaging signals in three or more imaging planes, based on the mode, to simultaneously capture both the interventional device and the anatomy targeted by the interventional device. In known ultrasound, X-planes use 2 imaging planes, such as 2 perpendicular planes, and capture only one of an interventional device or anatomy targeted by the interventional device. However, at S330, three or more imaging planes are used, and between the three or more imaging planes, the interventional device and the anatomy targeted by the interventional device are both simultaneously captured. For example, each of the three or more imaging planes may specifically intersect with one or both of the interventional device and/or the anatomy targeted by the interventional device.

At S340, the ultrasound probe is controlled to capture both the interventional device and the anatomy targeted by the interventional device, based on the emitted imaging signals in three or more planes. One of the interventional device and the anatomy targeted by the interventional device is captured in at least two of the three or more imaging planes, and the other of the interventional device and the anatomy targeted by the interventional device is simultaneously captured in least one of the three or more imaging planes. In embodiments, both of the interventional device and the anatomy targeted by the interventional device are simultaneously captured in two of the imaging planes, albeit not necessarily the same two imaging planes. In other embodiments, one or the other of the interventional device and the anatomy targeted by the interventional medical device are captured in one and only one of the imagine planes.

At S350, positions of the interventional device and the anatomy targeted by the interventional device are identified, based, for example, on the capture of reflected/returned imaging signals. Alternately, positions of the interventional device can be tracked from signals of a passive ultrasound sensor, or by other methods and mechanisms. Positions may be identified in a predetermined coordinate system, such as in a three-dimensional cartesian coordinate system with dimensions for width (X), height (Y) and depth (Z). A center of the coordinate system may be set at a fixed point in the space (volume) in or around the patient body.

In an embodiment, multiple different medical imaging systems may be registered to one another, so as to reflect commonality in viewpoints. Registration in this manner may involve setting coordinate systems of the different medical systems to reflect a common origin and common directionality dimensions.

At S360, a distance between the interventional device and anatomy targeted by the interventional device is determined and displayed. The distance may be determined in two dimensions, such as width (X)/height (Y), or may be determined in three dimensions such as width (X)/height (Y)/depth (Z).

At S370, a display is controlled to simultaneously display, in real-time, the interventional device and the anatomy targeted by the interventional device. A display may be or may include a screen on a television or on an electronic device such as a monitor. The monitor may be a monitor specifically provided with an ultrasound system, and may have settings specifically appropriate for visualizing imagery captured by the ultrasound system as well as related information such as information related to the captured imagery.

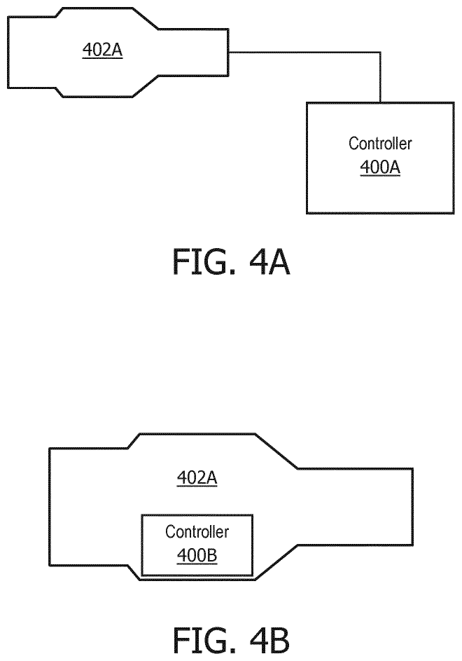

FIG. 4A illustrates a relationship between a probe and a controller for interventional medical device tracking, in accordance with a representative embodiment. In FIG. 4A, a probe 402A is separate from a controller 400A. The probe 402A is an imaging probe, and is controlled to activate imaging elements to emit imaging signals to generate imaging planes that intersect with tissue (e.g., in a patient body). The imaging elements may be transducer elements located on an imaging array. The probe 402A also captures interventional devices and anatomy targeted by the interventional devices in the imaging planes based on the response to the imaging signals (e.g., from the patient body). The probe 402A and controller 400A may communicate wirelessly or by wire. A controller 400A may include a processor 210, a main memory 220 and other elements from the computer system 200 shown in FIG. 2. A controller 400A may execute instructions to perform some or all of the software-based processes described herein, such as some or all of the aspects of the method shown in FIG. 3 herein. Such a controller 400A may be implemented by a computer such as a dedicated ultrasound system that controls a probe 402A and receives and processes imaging data from the probe 402A. Alternatively, a controller 400A may be a distributed subsystem of both the probe 402A and a separate computer that includes the processor 210 and main memory 220 (or other memory).

FIG. 4B illustrates another relationship between a probe and a controller for interventional medical device tracking, in accordance with a representative embodiment. In FIG. 4B, a probe 402A includes a controller 400B. That is, the controller 400B is a component of the probe 402A, and may include elements such as a processor 210 and a main memory 220. The probe 402B is also an imaging probe, and is controlled to activate imaging elements to emit imaging signals to generate imaging planes that intersect with tissue (e.g., in a patient body). The imaging elements may be transducer elements located on an imaging array. The probe 402B also captures interventional devices and anatomy targeted by the interventional devices in the planes based on the response of the tissue (e.g., in the patient body) to the imaging signals. A controller 400B in FIG. 4B may execute instructions to perform some or all of the software-based processes described herein.

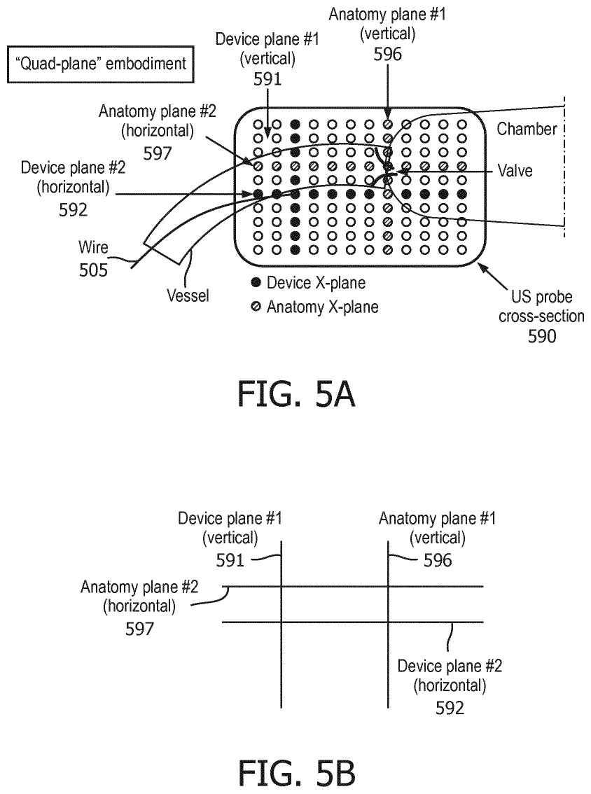

FIG. 5A illustrates a cross section of a probe for interventional medical device tracking, in accordance with a representative embodiment. FIG. 5A shows a “Quad-plane” embodiment in which one X-plane is tied to a device tip and one X-plane is tied to desired anatomy.

FIG. 5A shows the cross-section of the TEE (or other) ultrasound probe on the underlying cardiac anatomy. Active imaging planes are shown by lines of dots. In FIG. 5A, lines of dots in the third column from the left and sixth row from the top are tied to device position, which in turn is obtained from a device tracking method. Lines of dots in the eighth column from the left and fourth row from the top are tied to the desired anatomy, which in turn can be set by the user. Accordingly, in the embodiment of FIG. 5A, two active imaging planes are tied to the interventional device position, and two completely different active imaging planes are tied to the desired anatomy.

Specifically, in FIG. 5A, a wire 505 is overlaid on a vessel and exits the ultrasound probe 590 cross section to the left. A device plane #1 (vertical) 591 and a device plane #2 (horizontal) 592 correspond to the active imaging planes tied to the interventional device position. An anatomy plane #1 (vertical) 596 and an anatomy plane #2 (horizontal) 597 correspond to the active imaging planes tied to the desired anatomy.

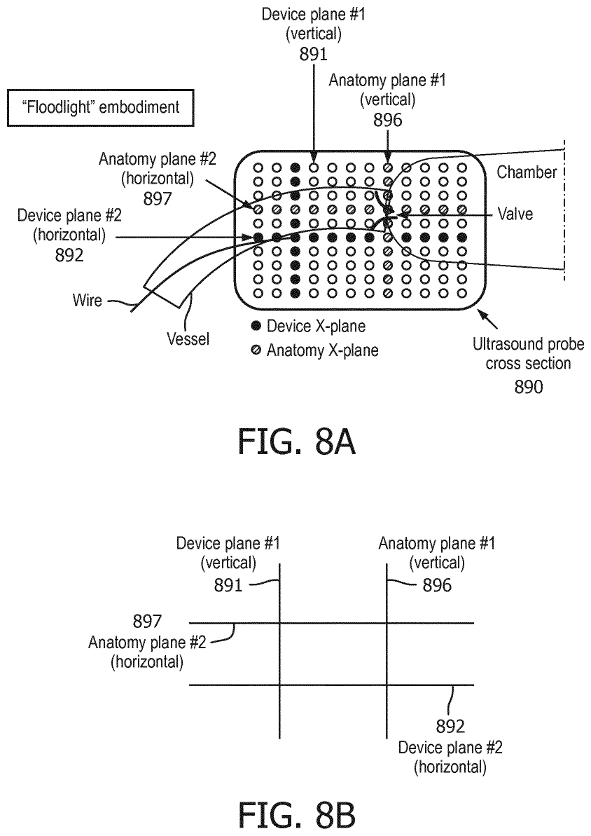

FIG. 5B illustrates a simplified view of imaging planes in the embodiment of FIG. 8A. In FIG. 5B, the device plane #1 (vertical) 591 and the anatomy plane #1 (vertical) 596 are shown as parallel vertical lines. Of course, the device plane #1 (vertical) 591 and the anatomy plane #1 (vertical) 596 do not have to be parallel to each other, or vertical, as these characteristics are used as a referential convenience. Similarly, device plane #2 (horizontal) 592 and anatomy plane #2 (horizontal) 597 are also shown as parallel lines, in this case horizontal lines. The device plane #2 (horizontal) 592 and anatomy plane #2 (horizontal) 597 also do not have to be parallel to each other, or horizontal, as these characteristics are also used only as a referential convenience.

However, the device plane #1 (vertical) 591 and the device plane #2 (horizontal) 592 are shown to be perpendicular, and this characteristic is accurately reflective of how these planes are best used to capture a targeted interventional device or anatomy targeted by an interventional device. Similarly, the anatomy plane #1 (vertical) 596 and anatomy plane #2 (horizontal) 597 are also shown to be perpendicular, and this characteristic is also accurately reflective of how these planes are best used to capture a targeted interventional device or anatomy targeted by an interventional device. Nevertheless, perpendicular planes do not have to be perfectly perpendicular, and may be substantially perpendicular while still working in their intended manner. Examples of substantially perpendicular planes may be intersecting planes with a smaller angle therebetween greater than 67.5 degrees, greater than 75 degrees, or greater than 85 degrees.

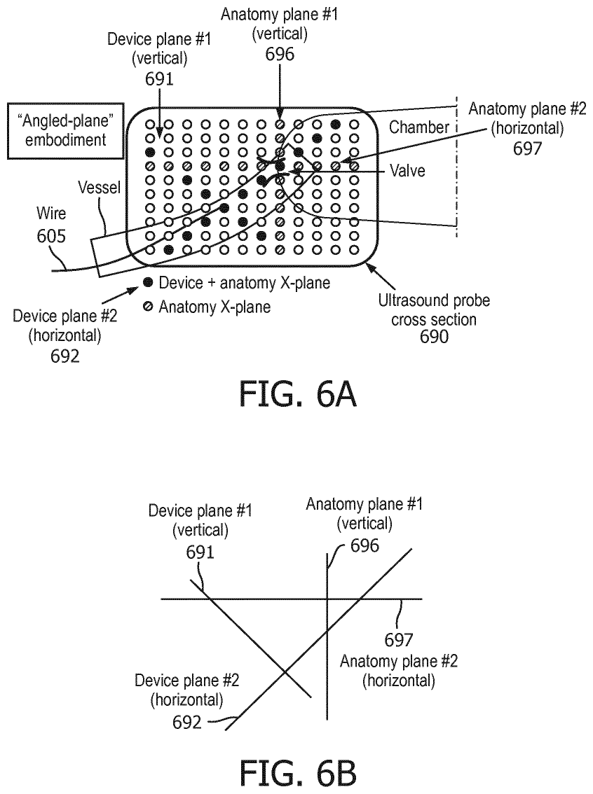

FIG. 6A illustrates another cross section of a probe for interventional medical device tracking, in accordance with a representative embodiment. FIG. 6A shows an “Angled-plane” embodiment in which one X-plane is tied to device and anatomy, and one X-plane is tied to anatomy.

FIG. 6A again shows the cross-section of the TEE (or other) ultrasound probe on the underlying cardiac anatomy. Active imaging planes are shown by lines of dots. In FIG. 6A, lines of dots in the eighth column from the left and fourth row from the top are tied to the desired anatomy, as in the embodiment of FIG. 5A and FIG. 5B. However, the lines of dots tied to the interventional device position are angled by being rotated about an axis to tilt. Accordingly, in the embodiment of FIG. 6A, two active imaging planes are again tied to the interventional device position, but are rotated about an axis to tilt, and two completely different active imaging planes are tied to the desired anatomy.

Specifically, in FIG. 6A, a wire 605 is again overlaid on a vessel and exits the ultrasound probe cross section 690 to the left. A device plane #1 (vertical) 691 and a device plane #2 (horizontal) 692 correspond to the active imaging planes tied to the interventional device position, but both are rotated about an axis to tilt. An anatomy plane #1 (vertical) 696 and an anatomy plane #2 (horizontal) 697 correspond to the active imaging planes tied to the desired anatomy. In the embodiment of FIG. 6A, the “device X-plane” is configured to image the plane containing the interventional device and the desired anatomy.

FIG. 6B illustrates a simplified view of imaging planes in the embodiment of FIG. 6A. In FIG. 6B, the device plane #1 (vertical) 691 and the device plane #2 (horizontal) 692 are rotated about an axis to tilt relative to the embodiment of FIG. 5A and FIG. 5B. However, the device plane #1 (vertical) 691 and the device plane #2 (horizontal) 692 are shown to be perpendicular, and may have the same characteristics as the similar planes in the embodiment of FIG. 5A and FIG. 5B other than their being rotated about an axis to tilt. The anatomy plane #1 (vertical) 696 and anatomy plane #2 (horizontal) 697 are also shown to be perpendicular, and may have the same or similar characteristics to the corresponding planes in the embodiment of FIG. 5A and FIG. 5B.

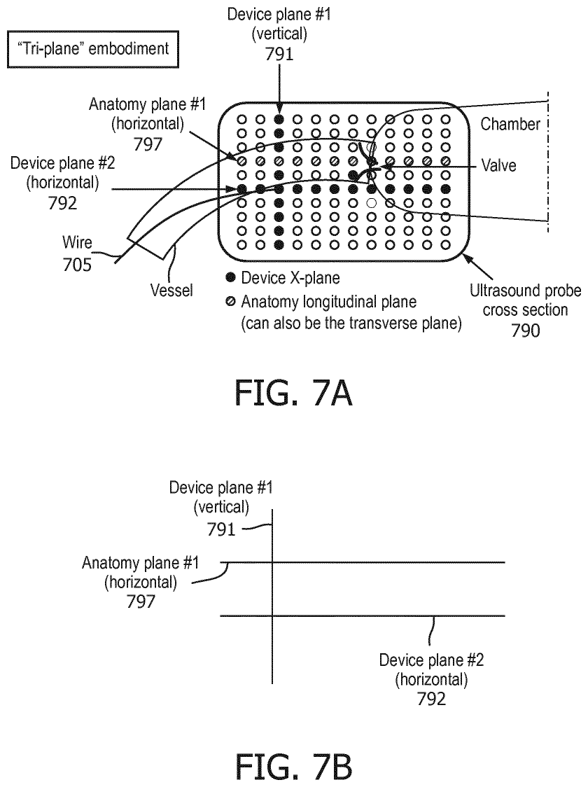

FIG. 7A illustrates another cross section of a probe for interventional medical device tracking, in accordance with a representative embodiment. FIG. 7A shows a “Tri-plane” embodiment in which one X-plane is tied to the interventional device tip and one long-axis plane is tied to anatomy.

FIG. 7A again shows the cross-section of the TEE (or other) ultrasound probe on the underlying cardiac anatomy. Active imaging planes are shown by lines of dots. In FIG. 7A, a single line of dots in the fourth row from the top are tied to the desired anatomy. Lines of dots in the third column from the left and sixth row from the top are tied to device position, the same as in the embodiment of FIG. 5A and FIG. 5B described previously.

Accordingly, in the embodiment of FIG. 7A, two active imaging planes are again tied to the interventional device position, but only one completely different active imaging plane is tied to the desired anatomy. In the embodiment of FIG. 7A, the anatomy imaging plane is a single plane, as opposed to a bi-plane, thereby resulting in slightly higher frame rate.

Specifically, in FIG. 7A, a wire 705 is again overlaid on a vessel and exits the ultrasound probe cross section 790 to the left. A device plane #1 (vertical) 791 and a device plane #2 (horizontal) 792 correspond to the active imaging planes tied to the interventional device position. A single anatomy plane #1 (horizontal) 797 corresponds to the active imaging plane tied to the desired anatomy. The anatomy plane #1 (horizontal) 797 is one and the only one imaging plane dedicated to the desired anatomy in the embodiment of FIG. 7A.

In an alternative embodiment, the one anatomy plane #1 (horizontal) 797 can be a short-axis imaging plane rather than a long-axis imaging plane. In still another alternative to the embodiment shown in FIG. 7A, a single X-plane may be assigned to anatomy, and a single plane assigned to the device.

FIG. 7B illustrates a simplified view of imaging planes in the embodiment of FIG. 7A. In the embodiment of FIG. 7B, the device plane #1 (vertical) 791 is perpendicular or substantially perpendicular to the device plane #2 (horizontal) 792, and the anatomy plane #1 (horizontal) 797 has no corresponding vertical anatomy plane.

FIG. 8A illustrates another cross section of a probe for interventional medical device tracking, in accordance with a representative embodiment.

FIG. 8A shows a “Floodlight”/“look ahead” embodiment in which the transverse plane of the interventional device X-plane is positioned ‘x’ mm ahead of the tip, to show the “upcoming” anatomy if the interventional device is pushed further.

FIG. 8A shows the cross-section of the TEE (or other) ultrasound probe on the underlying cardiac anatomy. Active imaging planes are shown by lines of dots. In FIG. 8A, lines of dots in the fourth column from the left and sixth row from the top are tied to device position, which in turn is obtained from a device tracking method. Thus, the imaging plane in the fourth column is adjusted based on movement of the interventional device and a current position of the interventional device. In other words, the imaging plane in the fourth column is set based on a trajectory of an intervention in progress, in order to look ahead to show the anatomy that will be encountered when the interventional device is moved further ahead. The current position refers to the position of the interventional device at the time the trajectory is set. Lines of dots in the eighth column from the left and fourth row from the top are tied to the desired anatomy, which in turn can be set by the user. Accordingly, in the embodiment of FIG. 8A, two active imaging planes are tied to the interventional device position, and two completely different active imaging planes are tied to the desired anatomy.

Specifically, in FIG. 8A, a wire 805 is overlaid on a vessel and exits the ultrasound probe 890 cross section to the left. A device plane #1 (vertical) 891 and a device plane #2 (horizontal) 892 correspond to the active imaging planes tied to the interventional device position. An anatomy plane #1 (vertical) 896 and an anatomy plane #2 (horizontal) 897 correspond to the active imaging planes tied to the desired anatomy.

Here, the transverse plane of the interventional device X-plane tied to the interventional device position is adjusted to image the region of tissue “just ahead” of the current device position. The adjusted transverse plane thereby shows which tissue the interventional device will encounter if the interventional device is pushed ahead further in the current direction. Current direction can be determined from the recent history of device positions.

FIG. 8B illustrates a simplified view of imaging planes in the embodiment of FIG. 8A. In FIG. 8B, the various planes are similar to those shown in the embodiment of FIG. 5B. The device plane #1 (vertical) 891 and the device plane #2 (horizontal) 592 are shown to be perpendicular or substantially perpendicular, and the anatomy plane #1 (vertical) 896 and anatomy plane #2 (horizontal) 897 are also shown to be perpendicular or substantially perpendicular. However, as noted above, the device plane #1 (vertical) 891 can be projected based on the position and directionality of the interventional tool, so that the device plane #1 (vertical) 891 can be automatically controlled using feedback from the historical movement and positioning of the interventional tool.

An example of projecting for the embodiments of FIG. 8A and FIG. 8B includes taking the angles of movement over time relative to a vertical axis, a horizontal axis, and a depth axis, particularly if the most recent movement is in a straight line or anything close to a straight line. In this example, the projecting can also take into account speed of movement, such as millimeters per second, in order to identify how far ahead of a current position to target for the anatomy plane #1 (vertical) 896.

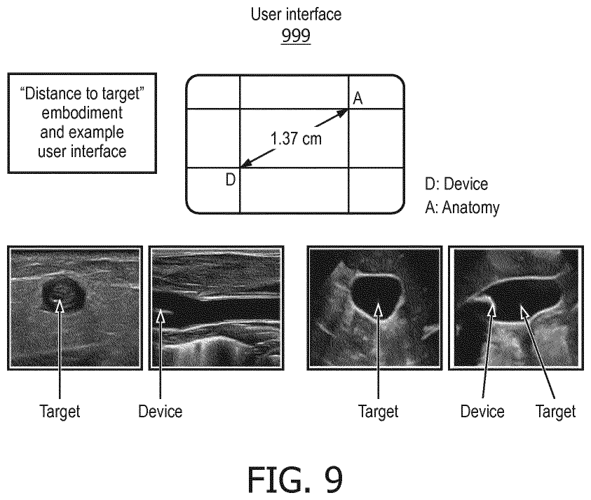

FIG. 9 illustrates views presented on a user interface for interventional medical device tracking, in accordance with a representative embodiment.

In FIG. 9, “Distance to target” embodiment: Display distance to anatomical target imaging plane on the interventional device X-plane.

At any time during a procedure, a “distance to target” can be calculated from the current device location and the desired anatomical target, and shown to the user in real-time. This is shown in FIG. 9 in conjunction with a sample user interface 999.

Accordingly, interventional medical device tracking enables selective use of different numbers of imaging planes in order to simultaneously capture both an interventional device and anatomy targeted by the interventional device. This provides visualization of tissue around a device and other quantitative navigation metrics, without losing sight of targeted anatomy.

Although interventional medical device tracking has been described with reference to several exemplary embodiments, it is understood that the words that have been used are words of description and illustration, rather than words of limitation. Changes may be made within the purview of the appended claims, as presently stated and as amended, without departing from the scope and spirit of interventional medical device tracking in its aspects. Although interventional medical device tracking has been described with reference to particular means, materials and embodiments, interventional medical device tracking is not intended to be limited to the particulars disclosed; rather interventional medical device tracking extends to all functionally equivalent structures, methods, and uses such as are within the scope of the appended claims.

For example,

Although the present specification describes components and functions that may be implemented in particular embodiments with reference to particular standards and protocols, the disclosure is not limited to such standards and protocols.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1.-15. (canceled)

16. A system for tracking an interventional medical device in a patient, comprising:
an imaging probe configured to activate imaging elements to emit imaging beams to generate three or more imaging planes within a field of view including a first imaging plane, a second imaging plane, and a third imaging plane perpendicular to the second imaging plane, to simultaneously capture an interventional medical device and anatomy targeted by the interventional medical device; and
a controller configured to control the imaging probe to simultaneously capture both the interventional medical device and the anatomy targeted by the interventional medical device, the interventional medical device and targeted anatomy being physically separated in a three-dimensional space, wherein the imaging probe is controlled to capture at least one of the interventional medical device and the anatomy targeted by the interventional medical device in at least two of the three or more imaging planes, and to capture the other of the interventional medical device and the anatomy targeted by the interventional medical device in at least one of the three or more imaging planes, wherein the controller includes a signal processor that processes image signals that simultaneously capture at least one of the interventional medical device and the anatomy targeted by the interventional medical device in at least two of the three or more imaging planes and the other of the interventional device and the anatomy targeted by the interventional device in at least one of the three or more imaging planes;
wherein controlling the imaging probe to capture the interventional medical device comprises automatically following the interventional medical device with the respective imaging plane(s) based on tracked positions of the interventional medical device determined by analyzing ultrasound signals received by a passive ultrasound sensor disposed on the interventional medical device as the imaging beams of the ultrasound probe sweep the field of view.

17. The system of claim 16,
wherein the imaging probe comprises a transesophageal echocardiography (TEE) ultrasound probe.

18. The system of claim 16 further comprising a display, and wherein the controller is further configured to control the display to simultaneously display in real-time the interventional medical device and the anatomy targeted by the interventional medical device.

19. The system of claim 16,
wherein the second imaging plane and third imaging plane are dedicated to the interventional medical device.

20. The system of claim 16,
wherein the second imaging plane and third imaging plane are dedicated to the anatomy targeted by the interventional medical device.

21. The system of claim 16, wherein the three or more imaging planes further includes a fourth imaging plane perpendicular to the first imaging plane.

22. The system of claim 21, wherein the second imaging plane and the third imaging plane are configured to capture the interventional medical device, and
the first imaging plane and the fourth imaging plane are configured to capture the anatomy targeted by the interventional medical device.

23. The system of claim 22, wherein the first imaging plane and the second imaging plane are substantially parallel, and
wherein the third imaging plane and the fourth imaging plane are substantially parallel.

24. The system of claim 16, wherein the second imaging plane and the third imaging plane are configured to capture both the interventional medical device and the anatomy targeted by the interventional medical device.

25. The system of claim 24, wherein the three or more imaging planes further includes a fourth imaging plane perpendicular to the first imaging plane.

26. The system of claim 25, wherein the controller is further configured to control the imaging probe to rotate the fourth imaging plane and first imaging plane about an axis to tilt the fourth imaging plane and first imaging plane relative to the second imaging plane and the third imaging plane.

27. The system of claim 21, wherein the fourth imaging plane is dedicated to the interventional medical device, and
wherein the fourth imaging plane is adjusted to image a region of the anatomy targeted by the interventional device projected based on movement of the interventional medical device and a current position of the interventional medical device (105).

28. A method for tracking an interventional medical device in a patient using the system of claim 16, the method, comprising:
emitting, by activating imaging elements controlled by the imaging probe, imaging signals to generate three or more imaging planes including the first imaging plane, the second imaging plane, and the third imaging plane perpendicular to the second imaging plane, to simultaneously capture the interventional medical device and anatomy targeted by the interventional medical device, the interventional medical device and targeted anatomy being physically separated in a three-dimensional space; and
simultaneously capturing the interventional medical device and the anatomy targeted by the interventional medical device, wherein the imaging probe is controlled to capture at least one of the interventional medical device and the anatomy targeted by the interventional medical device in at least two of the three or more imaging planes, and to capture the other of the interventional medical device and the anatomy targeted by the interventional medical device in at least one of the three or more imaging planes;
wherein controlling the imaging probe to capture the interventional medical device comprises automatically following the interventional medical device with the respective imaging plane(s) based on tracked positions of the interventional medical device determined by analyzing ultrasound signals received by the passive ultrasound sensor disposed on the interventional medical device as the imaging beams of the ultrasound probe sweep the field of view.

29. The method of claim 28, further comprising:
identifying, in a predetermined coordinate system, a position of the interventional medical device and a position of the anatomy targeted by the interventional medical device, and
producing a distance between the position of the interventional medical device and the position of the anatomy targeted by the interventional medical device.

30. The method of claim 28, further comprising:
simultaneously displaying the interventional medical device and the anatomy targeted by the interventional medical device based on the simultaneously capturing the interventional medical device and the anatomy targeted by the interventional medical device.
